(19) United States
(12) Patent Application Publication
(10) Pub. No.: US 2009/0171313 A1
(43) Pub. Date: Jul. 2, 2009

(54) IONTOPHORESIS DEVICE HAVING AN ACTIVE ELECTRODE UNIT

(76) Inventors: Akira Yamamoto, Kyoto (JP); Takehiko Matsumura, Tokyo (JP); Mizuo Nakayama, Tokyo (JP); Hidero Akiyama, Tokyo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/336,122

(22) Filed: Dec. 16, 2008

Related U.S. Application Data
(60) Provisional application No. 61/017,092, filed on Dec. 27, 2007.

(51) Int. Cl. A61N 1/30

(52) U.S. Cl. 604/501; 604/20

(57) ABSTRACT

An iontophoresis device transdermally administers an active agent, such as a drug ion, to a biological interface of an organism. The iontophoresis device includes a first electrode assembly having a first electrode member, which is electrically coupled to a terminal, of a main electric power source, having a first polarity that is the same polarity as that of a drug ion. The iontophoresis device includes a drug solution reservoir arranged in an electric field generated by the first electrode member and holding a drug, a counter electrode assembly electrically coupled to another terminal (of the main electric power source) having a second polarity that is opposite to the first polarity, and a vibrating portion having an ultrasonic oscillator for oscillating an ultrasonic wave and an ultrasonic vibrator vibrating due to the ultrasonic wave supplied from the ultrasonic oscillator. The ultrasonic vibrator is provided in the vicinity of the active electrode assembly.

### TECHNICAL FIELD

The present disclosure relates to an iontophoresis device having an active electrode unit. In particular but not exclusively, the present disclosure relates to an iontophoresis device for transdermally administering a drug ion by using iontophoresis, and an active electrode unit used therefor.

### BACKGROUND INFORMATION

An iontophoresis device uses iontophoresis for transdermally administering a drug ion to a surface of an organism, such as a skin or a mucous membrane on a desired portion of a body of a human or an animal. It is noted that, in some cases, iontophoresis is also referred to as iontophorese, an ion introducing method, an ion osmosis treatment, or the like.

A vibrator or an ultrasonic vibrator is provided on an electrode of some iontophoresis devices, such as described in Japanese Patent No. 2788307 and Japanese Patent Application Laid-open No. Hei 08-252329.

In the iontophoresis devices as described in the above-identified patent documents, a drug is provided between the skin and the electrode located on the drug administration side of the iontophoresis device. Therefore, the drug and the skin, and the drug and the electrode, are brought into contact with each other. Accordingly, there is a risk of a harmful substance being generated by an electrolytic reaction between the drug or water (which is a medium used for dissolving the drug) on the electrode, thereby causing scalding or inflammation on the skin. Further, a hydrogen gas or an oxygen gas generated by the electrolytic reaction may interfere with the contact between the electrode and the drug, thereby undesirably increasing a conductive resistance and reducing a transport ratio of the drug ions.

### BRIEF SUMMARY

According to a first aspect, an iontophoresis device includes: an active electrode assembly to deliver an active agent to first region of a biological interface in response to applied current; a counter electrode assembly coupled to the active electrode assembly; a vibration portion including an oscillator to generate an ultrasonic wave and at least one ultrasonic vibrator coupled to the oscillator and responsive to the ultrasonic wave to generate vibration, having a controllable vibration frequency and a controllable vibration duration, to be applied to a second region of the biological interface different from the first region; and a vibration absorption material that physically couples the vibration portion to the active electrode assembly, the vibration absorption material adapted to reduce transfer of the vibration from the at least one vibrator to the active electrode assembly so as to stabilize contact between the active electrode assembly and the first region of the biological interface.

According to another aspect, a method for an iontophoresis device includes: delivering, from an active electrode assembly of the iontophoresis device, an active agent to first region of a biological interface in response to applied current; delivering vibration having a controllable vibration frequency and a controllable vibration duration to a second region of the biological interface different from the first region without delivering more than a negligible amount of vibration to the first region; and reducing transfer of the vibration to the active electrode assembly so as to stabilize contact between the active electrode assembly and the first region of the biological interface.

According to a further aspect, an iontophoresis device to administer an active agent to a biological interface includes: a power source having a first terminal with a first polarity and a second terminal with a second polarity, the first polarity being opposite to the second polarity; an active electrode assembly having a first electrode member electrically coupled to the first terminal of the power source, the first polarity being same as a polarity of the active agent, and an active agent reservoir to contain the active agent and being arranged in an electric field generated by the first electrode member; a counter electrode assembly electrically coupled to the second terminal of the electric power source; and a vibration portion having an oscillator to provide an ultrasonic wave and at least one vibrator adapted to vibrate in response to the ultrasonic wave provided by the oscillator, wherein vibration generated by the vibrator has a controllable vibration frequency and a controllable vibration duration and is at least partially reduced from being transferred to the active electrode assembly.

According to a still further aspect, an iontophoresis device includes: an active electrode means for delivering an active agent to first region of a biological interface; means for generating and delivering vibration having a controllable vibration frequency and a controllable vibration duration to a second region of the biological interface different from the first region; and means for reducing transfer of the generated vibration to the active electrode means so as to stabilize contact between the active electrode means and the first region of the biological interface.

### BRIEF DESCRIPTION OF THE SEVERAL VIEWS OF THE DRAWINGS

Non-limiting and non-exhaustive embodiments are described with reference to the following drawings, wherein like reference numerals refer to like parts throughout the various views unless otherwise specified. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings

FIG. 1 is a perspective view of an iontophoresis device according to an embodiment.

FIG. 2 is a cross-sectional and schematic side view of one embodiment of the iontophoresis device of FIG. 1.

FIG. 3 is a cross-sectional and schematic side view of another embodiment of the iontophoresis device of FIG. 1.

FIG. 4 is an end view showing an example arrangement of an ultrasonic vibrator in the iontophoresis device of FIG. 1 according to one embodiment.

FIG. 5 is an end view of an embodiment in which an active electrode assembly is arranged annularly so as to surround a vibrating portion of the iontophoresis device of FIG. 1.

### DETAILED DESCRIPTION

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “membrane” means a layer, barrier or material, which may, or may not be permeable. Unless specified otherwise, membranes may take the form a solid, liquid or gel, and may or may not have a distinct lattice or cross-linked structure.

As used herein, the term “ion selective membrane” or similar means a membrane that is substantially selective to ions, passing certain ions while blocking passage of other ions. An ion selective membrane for example, may take the form of a charge selective membrane, or may take the form of a semi-permeable membrane.

As used herein and in the claims, the term “charge selective membrane” or similar means a membrane that substantially passes and/or substantially blocks ions based primarily on the polarity or charge carried by the ion. Charge selective membranes are typically referred to as ion exchange membranes, and these terms are used interchangeably. Charge selective or ion exchange membranes may take the form of a cation exchange membrane, an anion exchange membrane, and/or a bipolar membrane. Examples of commercially available cation exchange membranes include those available under the designators NEOSEPTA, CM-1, CM-2, CMX, CMS, and CMB from Tokuyama Co., Ltd. Examples of commercially available anion exchange membranes include those available under the designators NEOSEPTA, AM-1, AM-3, AMX, AHA, ACH and ACS also from Tokuyama Co., Ltd.

As used herein, the term “bipolar membrane” or similar means a membrane that is selective to two different charges or polarities. Unless specified otherwise, a bipolar membrane may take the form of a unitary membrane structure or multiple membrane structure. The unitary membrane structure may have a first portion including cation ion exchange material or groups and a second portion opposed to the first portion, including anion ion exchange material or groups. The multiple membrane structure (e.g., two film) may be formed by a cation exchange membrane attached or coupled to an anion exchange membrane. The cation and anion exchange membranes initially start as distinct structures, and may or may not retain their distinctiveness in the structure of the resulting bipolar membrane.

As used herein, the term “semi-permeable membrane” or similar means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein, the term “porous membrane” or similar means a membrane that is not substantially selective with respect to ions at issue. For example, a porous membrane is one that is not substantially selective based on polarity, and not substantially selective based on the molecular weight or size of a subject element or compound.

A used herein, the term “reservoir” or similar means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound.

The headings provided herein are for convenience only and do not interpret the scope or meaning of the embodiments.

In order to solve the above-described and other problems, a first embodiment provides an iontophoresis device for transdermally administering an active agent, such as an ionized drug. The iontophoresis device includes: an active electrode assembly having a first electrode member electrically coupled to a terminal (of an electric power source) having a first polarity that is the same polarity as that of an ionized drug, and a drug solution reservoir arranged in an electric field generated by the first electrode member and holding a drug solution including the drug; a counter electrode assembly electrically coupled to a terminal (of the electrical power source) having a second polarity that is opposite to the first polarity; an ultrasonic oscillator for oscillating an ultrasonic wave; and a vibrating portion having an ultrasonic vibrator which vibrates due to the ultrasonic wave supplied from the ultrasonic oscillator. With this structure, scald or inflammation on a biological interface (such as skin) coming into contact with the active electrode assembly can be prevented, and so the drug ion can be safely administered. Further, cavitation is caused in an inside of the skin by the ultrasonic vibrator, thereby making it possible to deteriorate a barrier performance of a stratum corneum. Thus, transdermal administration of the drug ion can be further promoted.

The active electrode assembly of one embodiment may further include: a first electrolyte solution reservoir electrically coupled to the first electrode member and holding an electrolyte solution; an ion exchange membrane of a second polarity sandwiching the first electrolyte solution reservoir between the first electrode member and itself and selectively allowing an ion of the second polarity to pass therethrough; and an ion exchange membrane of a first polarity sandwiching a drug solution reservoir between the ion exchange membrane of the second polarity and itself and selectively allowing an ion of the first polarity to pass therethrough. With this structure, it is possible not only to prevent scalding or inflammation on the biological interface coming into contact with the active electrode assembly, but also enables administration of the drug ion in a stable energized state. Therefore, the drug ion can be administered to an organism safely and efficiently.

The counter electrode assembly of one embodiment may further include: a second electrode member electrically coupled to the terminal of the electrical power source; a second electrolyte solution reservoir electrically coupled to the second electrode member and holding an electrolyte solution; an ion exchange membrane of a first polarity sandwiching the second electrolyte solution reservoir between the second electrode member and itself and selectively allowing an ion having a different electrical polarity from that of the second electrode member to pass therethrough; a third electrolyte solution reservoir arranged on an opposite side of the second electrolyte solution reservoir in the ion exchange membrane of the first polarity; and an ion exchange membrane of a second polarity sandwiching a third electrolyte solution reservoir between the ion exchange membrane of the first polarity and itself and selectively allowing an ion having the same electrical polarity as the second electrode member to pass therethrough. With this structure, it is possible not only to prevent scalding or inflammation on the biological interface coming into contact with the counter electrode assembly, but also enables administration of the drug ion in the more stable energized state. Therefore, the drug ion can be administered to the organism safely and efficiently.

Further in one embodiment, in the iontophoresis device, the ultrasonic vibrator may be arranged in the vicinity of or otherwise proximate to the active electrode assembly, such as in parallel thereto. With this structure, unlike in a case where the ultrasonic vibrator is arranged on, under, or in the active electrode assembly, the ultrasonic vibrator does not cause the active electrode assembly to vibrate. Accordingly, it is possible to prevent a contact state between the active electrode assembly and the skin from becoming unstable due to vibration. Further, unlike in the case where the ultrasonic vibrator is arranged on, under, or in the active electrode assembly, the active electrode assembly of one embodiment does not have to integrate or otherwise include a structure for transmitting vibration to the skin coming into contact with the active electrode assembly.

Further in one embodiment, the ultrasonic vibrator may be arranged annularly so as to surround the first electrode member. With this structure, the vibration generated by the ultrasonic vibrator can more reliably be transmitted to the biological interface coming into contact with the active electrode assembly, thereby enhancing the administration of the drug ion to the organism by the vibration.

Further in one embodiment, the ultrasonic vibrator may be detachably attached to the active electrode assembly. With this structure, in a case where the ultrasonic vibrator is mounted to the active electrode assembly, the ultrasonic vibrator can be handled with the active electrode assembly as one unit, and so operability for a user may be improved. Further, when the ultrasonic vibrator is not used, the ultrasonic vibrator can be detached from the active electrode assembly, thereby being capable of achieving reduction in weight.

Further in one embodiment, the iontophoresis device may include a separate vibration electric power source for supplying electricity to the ultrasonic oscillator coupled to the ultrasonic vibrator. With this structure, as compared to another embodiment where the electric power source supplies electricity to the active electrode assembly, the counter electrode assembly, and also the vibrating portion, a size of the power source can be made smaller. Further, the active electrode assembly, the counter electrode assembly, and the vibrating portion can be operated independently from one another in the embodiment where the vibration power source and the power source are separately provided.

Further in one embodiment, the ultrasonic vibrator may be mounted to the active electrode assembly through a vibration absorption material. With this structure, it is possible to reduce vibration from the ultrasonic vibrator from transferring to the active electrode assembly, thereby preventing the contact state between the active electrode assembly and the biological interface from becoming unstable due to vibration.

Further in one embodiment, the iontophoresis device may include a control portion for controlling electricity supplied from the electric power source to the active electrode assembly, the counter electrode assembly, and the ultrasonic oscillator. With this structure, electricity supplied to the active electrode assembly, the counter electrode assembly, and the ultrasonic oscillator can be properly controlled depending on factors such as but not limited to the frequency and duration of vibration during administration of the drug, frequency and duration of drug administration, and so forth. Consequently, a drug ion can be administered to the organism more safely and efficiently.

According to another embodiment, an active electrode unit in an iontophoresis device for administering a drug ion to an organism includes: an active electrode assembly having a first electrode member electrically coupled to a terminal of a second polarity that is opposite to the first polarity, and a drug solution reservoir arranged in an electric field generated by the first electrode member and holding the drug solution; an ultrasonic oscillator for oscillating an ultrasonic wave; and a vibrating portion which vibrates due to the ultrasonic wave supplied from the ultrasonic oscillator.

According to an embodiment, the scalding or inflammation on the biological interface coming into contact with the active electrode assembly can be prevented or otherwise reduce, and so the drug ion can be safely administered to the organism. Further, the ultrasonic vibrator causes cavitation inside of the biological interface, thereby deteriorating the barrier performance of the stratum corneum of the skin, for example. As a result, the transdermal administration of the drug ion can be improved.

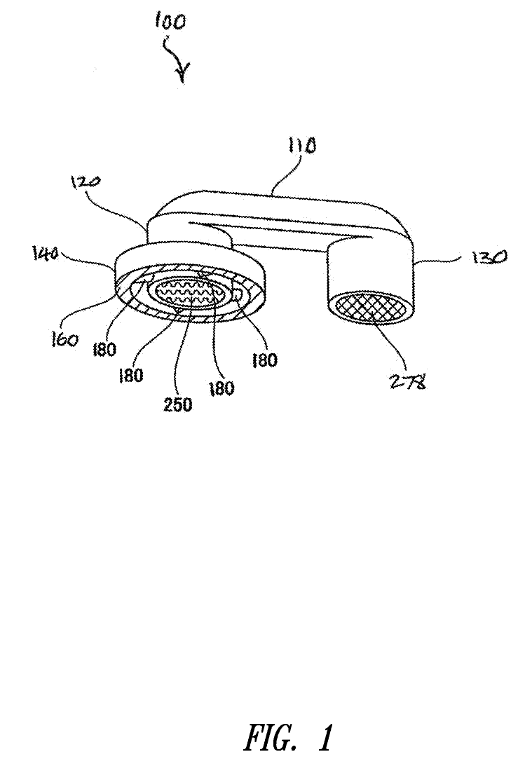

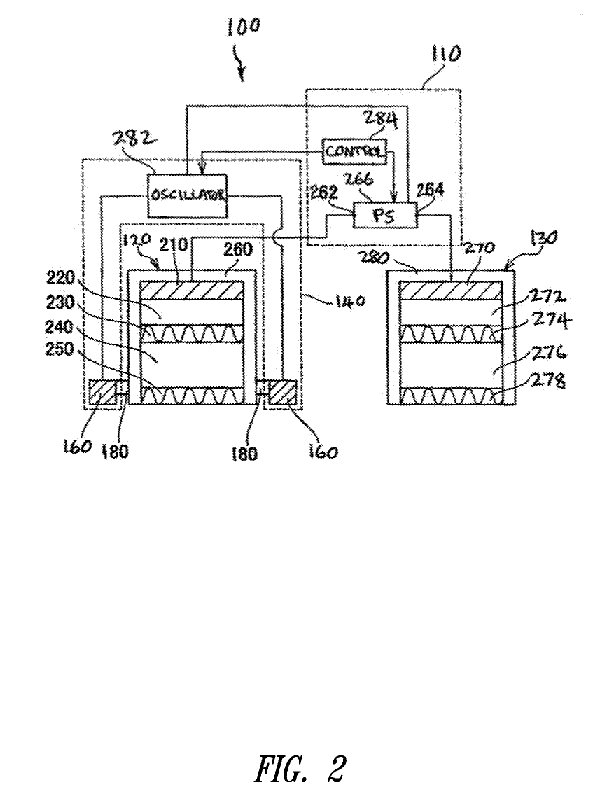

FIG. 1 is a perspective view of an iontophoresis device 100 according to one embodiment. FIG. 2 is a cross-sectional and schematic side view of the iontophoresis device 100 of FIG. 1. The iontophoresis device 100 shown in FIGS. 1 and 2 includes a device main body 110, an active electrode assembly 120, a counter electrode assembly 130, and a vibrating portion 140 provided proximate to the active electrode assembly 120. In the following description, a positive (+) polarity and a negative (−) polarity are respectively referred to as a first polarity and as a second polarity, unless otherwise specified below.

The active electrode assembly 120 of one embodiment includes, from the device main body 110 side towards a side proximate to a biological interface (for example skin in a case where the active electrode assembly 120 is mounted to the skin) a first electrode member 210, a first electrolyte solution reservoir 220, an ion exchange membrane 230 having a second polarity, a drug solution reservoir 240, and an ion exchange membrane 250 having a first polarity. An upper surface and a side surface of the active electrode assembly 120 are covered with a container 260 or other suitable housing. The first electrode member 210 is electrically coupled to a terminal 262 (having the first polarity) of a main electric power source 266 built in or otherwise included in the device main body 110.

The first electrolyte solution reservoir 220 is electrically coupled to the first electrode member 210 and holds an electrolyte solution. The electrolyte solution is obtained by dissolving a compound that is both oxidized and reduced easily and that has an oxidation reaction potential lower than that of water. The ion exchange membrane 230 of the second polarity and the first electrode member 210 sandwich the first electrolyte solution reservoir 220. The ion exchange membrane 230 of the second polarity selectively allows an ion having the second polarity to pass therethrough.

The drug solution reservoir 240 holds a drug solution including a drug ion. An example drug ion is an ion having the first polarity and having a drug effect, with the drug ion being one of an anion and a cation obtained through ion dissociation of the drug. The ion exchange membrane 250 of the first polarity and the ion exchange membrane 230 of the second polarity sandwich the drug solution reservoir 240. The ion exchange membrane 250 of the first polarity selectively allows the ion of the first polarity to pass therethrough.

The counter electrode assembly 130 includes, from the device main body 110 side towards the biological interface side, a second electrode member 270, a second electrolyte solution reservoir 272, an ion exchange membrane having the first polarity 274, a third electrolyte solution reservoir 276, and an ion exchange membrane having the second polarity 278. The second electrode member 270 is coupled to a terminal 264 (having the second polarity) of the main electric power source 266 built in or otherwise included in the device main body 110. An upper surface and a side surface of the counter electrode assembly 130 are covered with a container 280 or other suitable housing.

The second electrolyte solution reservoir 272 is electrically coupled to the second electrode member 270 and holds an electrolyte solution. The ion exchange membrane 274 of the first polarity and the second electrode member 270 sandwich the second electrolyte solution reservoir 272. The ion exchange membrane 274 of the first polarity selectively allows the ion of the first polarity to pass therethrough.

The third electrolyte solution reservoir 276 is arranged on an opposite side of the second electrolyte solution reservoir 272 relative to the ion exchange membrane 274 of the first polarity, and holds an electrolyte solution. The ion exchange membrane 278 of the second polarity and the ion exchange membrane 274 of the first polarity sandwich the third electrolyte solution reservoir 276. The ion exchange membrane 278 of the second polarity selectively allows the ion of the second polarity to pass therethrough.

Like the electrolyte solution held by the first electrolyte solution reservoir 220 in the active electrode assembly 120, the electrolyte solution held by the second electrolyte solution reservoir 272 and by the third electrolyte solution reservoir 276 are prepared by dissolving a compound that is both oxidized and reduced easily and that has an oxidation reduction potential lower than that of water.

The vibrating portion 140 of one embodiment includes an ultrasonic oscillator 282 for oscillating or otherwise supplying an ultrasonic wave and an ultrasonic vibrator 160 that is vibrated by the ultrasonic wave supplied from the ultrasonic oscillator 282. At least the ultrasonic vibrator 160 of the vibrating portion 140 is coupled in one embodiment to the active electrode assembly 120 via vibration absorption materials 180, and may be arranged annularly in one embodiment so as to at least partially surround the active electrode assembly 120. In this embodiment, the active electrode assembly 120 having the first electrode member 210 and the drug solution reservoir 240, the ultrasonic oscillator 282, and the ultrasonic vibrator 160 constitute the active electrode unit.

A control portion 284 controls electricity supplied from the main electric power source 266 to the ultrasonic vibrator 160 coupled to the active electrode assembly 120, the counter electrode assembly 130, and the ultrasonic oscillator 282. In this case, the control portion 284 may operate to control these various components according to a program stored therein. For example in one embodiment, the control portion 284 includes a processor and a computer-readable medium (such as a memory) storing computer-readable instructions (such as a computer program) that are executable by the processor to perform control operations.

In administering the drug ion to an organism by using the iontophoresis device 100 shown in FIGS. 1 and 2, at least an outer surface (the surface represented with wavy lines in FIG. 1) of the ion exchange membrane 250 of the first polarity of the active electrode assembly 120 and at least one surface (the shaded surface in FIG. 1) of the ultrasonic vibrator 160 of the vibrating portion 140 are brought into contact with an administration object site (e.g., an area of a biological interface) of the organism. Further, at least an outer surface (the cross-hatched surface in FIG. 1) of the ion exchange membrane 278 of the second polarity of the counter electrode assembly 130 is brought into contact with a periphery of the administration object site of the organism or a site connected to the administration object site of the organism.

As described above, in a state where the active electrode assembly 120 and the counter electrode assembly 130 of the iontophoresis device 100 are brought into contact with skin, for example, when electricity for applying iontophoresis is supplied (voltage is applied) from the main electric power source 266 to the first electrode member 210 and the second electrode member 270, a current flows between the first electrode member 210 and the second electrode member 270 through the skin, thereby realizing an energized state or completed electrical circuit.

For a case where the drug ion is an anion (as an example), a specific structure of the iontophoresis device 100 shown in FIGS. 1 and 2 will be described. In this case, the first polarity is negative (−) and the second polarity is positive (+). Accordingly, the first electrode member 210 of the active electrode assembly 120 is a cathode and the second electrode member 270 of the counter electrode assembly 130 is an anode. Further for the ion exchange membrane 230 of the second polarity in the active electrode assembly 120, a cation exchange membrane is used, and for the ion exchange membrane 250 of the first polarity, an anion exchange membrane is used. Further, for the ion exchange membrane 274 of the first polarity in the counter electrode assembly 130, an anion exchange membrane is used, and for the ion exchange membrane of the second polarity 278, a cation exchange membrane is used.

The iontophoresis device 100 shown in FIGS. 1 and 2 exerts the following operational effect in the energized state. That is, in the active electrode assembly 120, the drug ion included in the drug solution held by the drug solution reservoir 240 moves due to electrophoresis to an opposite side (the biological interface side) of the first electrode member 210 serving as a cathode, and passes through the ion exchange membrane 250 of the first polarity, which is provided on the biological interface side of the drug solution reservoir 240. The drug ion comes into contact with the biological interface, for example skin, to quickly permeate into the skin.

On the other hand, a cation in the organism does not pass through the ion exchange membrane of a first polarity 250 to move to the drug solution reservoir 240 side. Accordingly, the drug ion can be introduced to the organism by iontophoresis in the stable energized state. Further, a cation that forms a pair with the drug ion that is an anion and included in the drug solution reservoir 240 moves to the first electrode member 210 side, and so the cation passes through the ion exchange membrane 230 of the second polarity to move to the first electrolyte solution reservoir 220 side. Accordingly, in the energized state, an ion balance of the drug solution reservoir 240 is not thrown out, and so a change in pH does not easily occur. Accordingly, a conductive resistance is less prone to increase, and so a reduction in transportation efficiency of the drug ion can be suppressed.

In the counter electrode assembly 130, a compound dissolved in the electrolyte solution held by the third electrolyte solution reservoir 276 is a compound having an oxidation reaction potential lower than that of water. Therefore, in the second electrode member 270 serving as an anode, the electrolytic reaction of water does not occur. Accordingly, bubbles (oxygen gas) that would have been generated by the electrolytic reaction of water do not interfere with contact between the second electrode member 270 and the electrolyte solution held by the third electrolyte solution reservoir 276, thereby making it possible to prevent the increase of conductive resistance.

In a case where the drug ion is a cation, the first polarity is positive (+) and the second polarity is negative (−). Accordingly in the iontophoresis device 100 shown in FIGS. 1 and 2, the electrical polarities of the first electrode member 210 and the second electrode member 270 are inverted. Further, the types of the ion exchange membrane 230 of the second polarity and ion exchange membrane 250 of the first polarity, and the ion exchange membrane 274 of the first polarity and ion exchange membrane 278 of the second polarity 278 (ion selectivities) are respectively inverted with each other.

When administration of the drug ion to the organism is started by using the iontophoresis device 100 shown in FIGS. 1 and 2, electricity is supplied from the main electric power source 266 built in the device main body 110 of the iontophoresis device 100 to the ultrasonic oscillator 282 of the vibrating portion 140 through the control portion 284. The ultrasonic oscillator 282 converts the supplied electricity to a high-frequency voltage and the high-frequency voltage is applied to the ultrasonic vibrator 160. The ultrasonic vibrator 160 mechanically vibrates due to the high-frequency voltage, and ultrasonic vibration is transmitted to the biological interface brought into contact with the ultrasonic vibrator 160. Due to the ultrasonic vibration, cavitation occurs in the inside of the biological interface. The cavitation refers to a myriad of negative-pressure bubbles (cavities) generated at a cellular level. When the bubbles disappear, extremely large energy is generated locally. The energy acts on the stratum corneum to reduce the barrier performance thereof. As a result of the decrease in barrier performance, the transdermal absorption of the drug ion increases.

In this case, at least the ultrasonic vibrator 160 of the vibrating portion 140 is coupled to the active electrode assembly 120 by the vibration absorption materials 180. Accordingly, vibration from the ultrasonic vibrator is not directly transmitted to the active electrode assembly 120 side, and so vibration of the active electrode assembly 120 can be suppressed. Therefore, it is possible to prevent a contact state between the active electrode assembly 120 and the biological interface from becoming unstable as a result of vibration. Further, in one embodiment of the iontophoresis device 100, the ultrasonic vibrator 160 is arranged annularly so as to at least partially surround the active electrode assembly 120. Accordingly, the ultrasonic vibration generated by the ultrasonic vibrator 160 can be positively transmitted to the biological interface coming into contact with the active electrode assembly 120.

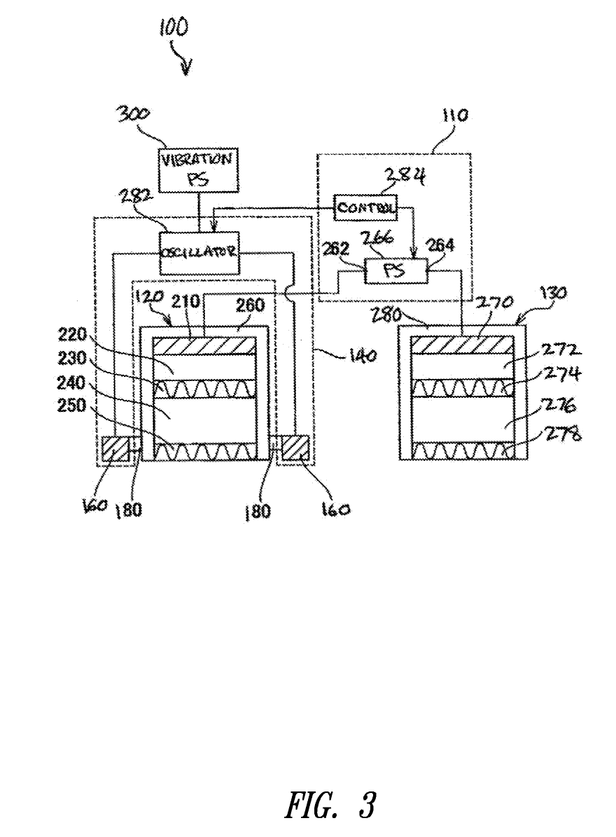

FIG. 3 is a schematic side view of the iontophoresis device 100 according to another embodiment, wherein similar elements are labeled the same as in FIG. 2. The iontophoresis device 100 shown in FIG. 3 includes, in addition to the structure of FIG. 2, a vibration electric power source 300 for supplying electricity to the ultrasonic oscillator 282 coupled to the ultrasonic vibrator 160. With this structure, the main electric power source 266 is sufficient for supplying electricity to the first electrode member 210 of the active electrode assembly 120 and to the second electrode member 270 of the counter electrode assembly 130. The main electric power source 266 is not used to supply electricity to the ultrasonic oscillator 282. Thus, the size of the main electric power source 266 can be made smaller. That is, the active electrode assembly 120 and the counter electrode assembly 130, and the vibrating portion 140, are separately provided with the main electric power source 266 and the vibration electric power source 300, respectively. Accordingly, the active electrode assembly 120 and the counter electrode assembly 130, and the vibrating portion 140 can be operated individually or independently of one another.

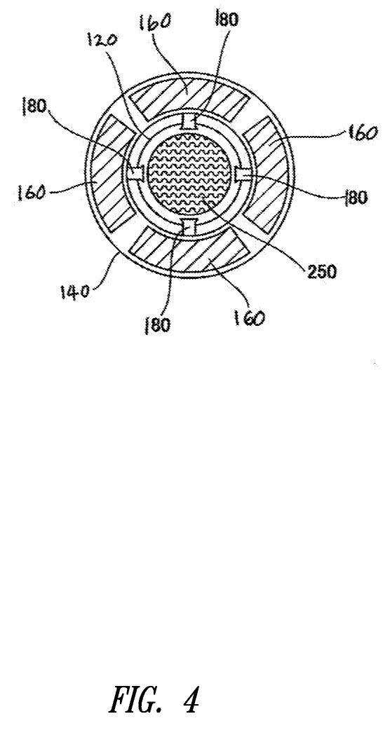

FIG. 4 is an end view showing another arrangement of the ultrasonic vibrator 160 in the iontophoresis device 100 according to one embodiment. As shown in FIG. 4, a plurality of the ultrasonic vibrators 160 may be provided so as to vibrate at a predetermined vibration mode. In one example of a vibration mode, all the plurality of the ultrasonic vibrators 160 may vibrate in the same direction at the same time. In another alternative or additional vibration mode, a first pair of ultrasonic vibrators 160 opposed to each other may vibrate in the same phase while a second pair of the ultrasonic vibrators 160 opposed to each other may vibrate at reverse phases relative to the first pair. Further, in another example vibration mode, the ultrasonic vibrators 160 may vibrate by shifting the phase clockwise or counterclockwise in a sequential order or other type of order. Other vibration modes may be provided. In one embodiment the control portion 284 can provide signals to independently control the vibration phase of each of the vibrators 160.

As explained previously above, the control portion 284 may be used in one embodiment to control the frequency and duration of the vibrator(s) 160 during administration of the drug. Thus, as one example, the vibration frequencies and/or duration of vibration of individual ones of the vibrators 160 can be controlled by the control portion 284 by providing control signals to the oscillator 282 for each respective vibrator 160. Alternatively or additionally, the vibration frequencies and/or duration of vibration of all of the vibrators 160 can be controlled to be the same.

As yet another example, the vibration frequency (of all or individual ones of the vibrators 160) and/or vibration duration (of all or individual ones of the vibrators 160) can be controlled by the control portion 284 so as to vary over time. For instance, the variable or constant frequencies of vibration and duration thereof might be applied intermittently or differently over time during administration of the drug. As an illustration, the vibration frequency might be higher and the vibration duration might last longer during the early stages of the drug administration, and then stop or pause intermittently during the middle stages of the drug administration, and then have lower vibration frequency and shorter vibration duration during the latter stages of the drug administration.

As still another example, the vibration frequency (of all or individual ones of the vibrators 160) and/or vibration duration (of all or individual ones of the vibrators 160), including any intermittent pauses as described above, can be controlled by the control portion 284 in a random or pseudo-random manner. In such an embodiment, therefore, the vibration frequency and/or duration, and/or any other timing factor (such as intermittent pauses in the vibration) may be random or pseudo-random. In one embodiment, the control portion 284 can be provided with a random number generator to provide this randomness, such that the output of the random number generator is used to control the timing or other generation of control signals to control the oscillator 282.

As still another example, the control portion 284 can be adapted such that the vibration and drug administration are influenced by each other timing-wise. For instance, the control portion 284 can be adapted to provide signals to the oscillator 282 such that the vibrations are synchronized with the drug administration—when the drug is being administered, the oscillator 282 is causing the vibrators 160 to vibrate at the same time. In other embodiments, the vibration and drug administration need not necessarily be synchronized with each other (e.g., may be asynchronous). For instance, it may be desirable in some instances to begin vibration before the drug is initially administered, or to not begin vibration until after the drug has been administered. In other situations, it may be desirable to continue vibration even after administration of the drug has completed (e.g., the drug reservoirs have been emptied), or to cease vibration before administration of the drug has fully completed.

As still another example, the intensity or strength of vibration can be controlled by the control portion 284. The intensity/strength of vibration can thus be controlled depending on factors such as the sensitivity of the patient, the type of drug being administered, the body location where the drug is being administered, the amount of cavitation desired, the hydration level of the biological interface, and so forth. As an illustration, the vibrations can be controlled by the control portion 284 so as to be minimal in intensity/strength, such that a patient with particularly sensitive nerves does not feel (or minimally feels) the vibration, as compared to another patient that may be less-bothered by vibration sensations.

The various examples above of controlling the phase, frequency, duration, intensity/strength, randomness, timing aspects, and/or other characteristic of the vibration can be applied differently for individual ones of the vibrators 160, and/or for a subset of the vibrators 160, and/or for all of the vibrators 160 as a whole.

Further, as shown in FIG. 4, the vibrating portion 140 including the ultrasonic vibrators 160 is detachably attached to the active electrode assembly 120 by fitting thereto (at four positions) the vibration absorption materials 180. With this structure, in a state where the vibrating portion 140 is mounted to the active electrode assembly 120, the vibrating portion 140 can be handled with the active electrode assembly 120 as one unit, and so operability for a user is improved. In addition, when the vibrating portion 140 is not used, the vibrating portion 140 can be detached, to thereby achieve a reduction in weight.

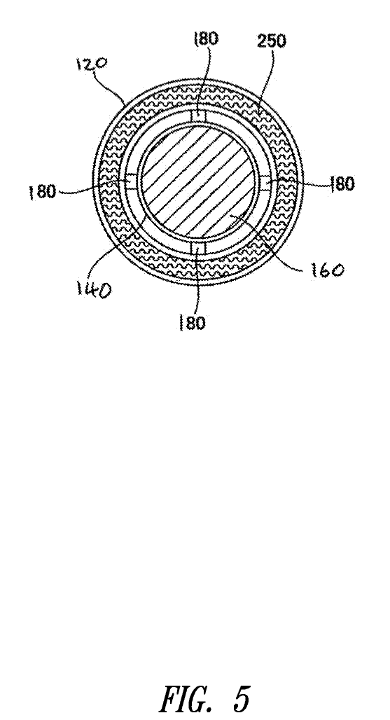

FIG. 5 shows an example arrangement of the iontophoresis device 100 in which the active electrode assembly 120 is configured or otherwise arranged annularly so as to at least partially surround the vibrating portion 140. As shown in FIG. 5, the active electrode assembly 120 is joined or otherwise coupled to the vibrating portion 140 by the vibration absorption materials 180. With this structure, in the vicinity of the biological interface that is allowed to vibrate by the ultrasonic vibrator 160, the drug ion can be administered from the active electrode assembly 120 to the organism. Accordingly, the administration of the drug ion to the organism by vibration can be enhanced.

The vibrating portion 140 of FIG. 5 can be embodied as a single vibrator that is annularly surrounded by the active electrode assembly 120, or may be embodied as a plurality of individual vibrators that is annularly surrounded by the active electrode assembly 120. Whether embodied as a single vibrator or as a plurality of individual vibrators, an embodiment of the vibrating portion 140 of FIG. 5 can be controlled by the control portion 284 in terms of phase, frequency, duration, timing aspects, intensity/strength, randomness, etc.

In the embodiments of FIGS. 4-5, it is shown that the ion exchange membrane 250 is placed into contact with a first region of a biological interface different from a second region of the biological interface that is placed into contact with the ultrasonic vibrator(s) 160. This configuration/arrangement enables the ultrasonic vibrator(s) 160 to apply the vibration to the second region, while the vibration absorption materials 180 reduces the direct transfer of the vibration from the ultrasonic vibrator(s) 160 to the active electrode assembly 120. The reduction of vibration on the active electrode assembly 120 enables the contact between the ion exchange membrane 250 and the first region to remain stable.

In the embodiments as shown in FIGS. 1 to 5, a voltage used for applying the iontophoresis can be a direct current (DC) voltage of about 0 to 100 V, for instance. For example, a pulse voltage may be applied in a case wherein the iontophoresis device 100 is used as low-frequency therapy equipment. Alternatively or additionally, the voltage may gradually be increased or reduced. A current flowing through a body falls within a range of 0.01 to 5 mA, for instance. However, the current can be controlled by the control portion 284 to such a degree that no pain or heat is given to a patient by increasing or decreasing the current, taking into account factors such as areas of the first electrode member 210 and the second electrode member 270, an administration position, an individual difference between patients, and the like.

Further, examples of the drug ion applied by the iontophoresis device 100 may include but are not limited to the following positively charged drug ions: anesthetics (such as procaine hydrochloride and lidocaine hydrochloride), gastrointestinal disease drugs (such as carnitine chloride), skeletal muscle relaxants (such as vecuronium bromide), and antibiotics (such as tetracycline-based preparations, kanamycin-based preparations, and gentamicin-based preparations). Examples of negatively charged drug ions may include but are not limited to: vitamin (V) preparations (such as VB₂, VB₁₂, VC, VE, and folic acid), adrenocortical hormones (such as hydrocortisone-based aqueous preparations, dexamethasone-based aqueous preparations, and prednisolone-based aqueous preparations), and antibiotics (such as penicillin-based aqueous preparations and chloramphenicol-based aqueous preparations).

Further, the main electric power source 266 and the vibration electric power source 300 are not limited to an embodiment where these power sources are integrated into the device main body 110. For example, the main electric power source 266 and/or the vibration electric power source 300 may be devices such as a battery, a constant current voltage device, a constant voltage/constant current device (galvano device) that may be integrated in the device main body 110 or be coupled separately therefrom. Further, for the vibration absorption materials 180, for example, a rubber pad or other suitable vibration absorption material may be used.

In one embodiment, the vibration absorption materials 180 may be in the form of foam pads.

In one embodiment, the vibration absorption materials 180 may be in the form of one or more springs having one end coupled to the ultrasonic vibrator 160 and another end coupled to the active electrode assembly 120.

In one embodiment, the vibration absorption materials 180 may be structured in a manner generally similar to shock absorbers, such that these shock absorbers are coupled between the ultrasonic vibrator(s) 160 and the active electrode assembly 120 to absorb vibrations. The shock absorber structures for the vibration absorption materials 180 according to various embodiments may be based on, but not be limited to, the following:
Hysteresis (somewhat analogous to a “memory” of a material—if pressure is applied to rubber disks, the rubber disks tend to return to their normal uncompressed state, as the pressure is relieved) of structural material, for example the compression of rubber disks, stretching of rubber bands and cords, bending of steel springs, or twisting of torsion bars. Hysteresis thus involves the tendency for otherwise elastic materials to rebound with less force than was required to deform them.
Dry friction by using disks (leather or some synthetic material) at a pivot of a lever, with friction forced by springs. A feature of such a technique is its mechanical simplicity—the degree of damping can be adjusted by tightening or loosening one or more screws or fitting that clamps the disks.
Solid state, tapered chain shock absorbers, using one or more tapered, axial alignment(s) of granular spheres (which may be made of metals such as nitinol or made of some other suitable material) in a casing. These granular materials are adapted to absorb shock/vibration.
Fluid friction, for example the flow of fluid through a narrow orifice (hydraulics). With this type of shock absorber, an internal valve may be used such that the shock absorber is made relatively soft to compression (allowing a soft response to a bump) and relatively stiff to extension. Further, a series of internal valves controlled by springs can change the degree of stiffness according to the velocity of the impact or rebound. According to some embodiments, the tuning of the shock absorber, via control of the internal valve(s), may be performed manually through manual adjustment of a dial or other adjustment element provided for the shock absorber. In other embodiments, the internal valves may be adjustable by the user using buttons or other user interface with the control portion 284, which is in turn coupled to the shock absorber and/or its related components to enable the internal valves to be adjusted. In yet other embodiments, adjustment of the internal valves can be performed by the control portion 284 with minimal or no input required from the user. For example, this control may be provided dynamically via the control portion 284 (such as via a computer program) in response to sensors that are adapted to sense the level of vibration being produced by the vibrator(s) 160. In yet another embodiment, a magneto-rheological damper may be used, which changes its fluid characteristics through an electromagnet.
Compression of a gas, for example pneumatic shock absorbers, which can act like springs as the air pressure is building to resist the force being applied. Once the air pressure reaches a maximum or other threshold, air dashpots act like hydraulic dashpots. Air dashpots may be combined with hydraulic damping to reduce bounce, for example, to provide a shock absorber somewhat analogous to “oleo struts.”
Magnetic effects—One embodiment may provide shock absorbers in the form of eddy current dampers, which are dashpots constructed out of a magnet inside of a non-magnetic, electrically conductive tube.
Inertial resistance to acceleration, for example shock absorbers that damp bounce with no external moving parts. These shock absorbers include a spring-mounted weight inside a vertical cylinder and are similar to, yet much smaller than versions of the tuned mass dampers used on tall buildings.
Composite hydropneumatic devices that combine in a single device spring action and shock absorption.
Shock absorbers combined with composite pneumatic springs.

Further, for an electrode material of the first electrode member 210 and the second electrode member 270, a suitable material (for example, a conductive material such as carbon or platinum) may be used, with the particular material being selected according to a desired property of the drug ion.

In addition, as described above, a solution prepared by dissolving a compound that is both oxidized and reduced easily and has an oxidation reduction potential lower than that of water, as compared to the electrolytic reaction of water (oxidation and reduction reactions of water), may be used for the electrolyte solution in each of: the first electrolyte solution reservoir 220 of the active electrode assembly 120; and the second electrolyte solution reservoir 272 and third electrolyte solution reservoir 276 of the counter electrode assembly 130. Examples of the solution include but are not limited to: a mixture solution of ferrous sulfate (FeSO₄) and ferric sulfate [Fe₂(SO₄)₃]; a sodium ascorbate solution; and a mixture solution of lactic acid and sodium fumarate. The electrolyte solution may be held in such a manner that the electrolyte solution is impregnated into a gel or a desired medium (such as a gauze or a water-absorption polymer material). Alternatively, the electrolyte solution may be held as it is (solution type).

Any desired anion exchange membrane may be used including but not limited to one having a quaternary ammonium group at a side chain of a polymer, and any desired cation exchange membrane may be used including but not limited to one having a sulfonic group at a side chain of a polymer. Those membranes may be appropriately combined depending on, for example, the kinds of the drug ions that are desired.

Further, the ultrasonic oscillator 282 may be an oscillator of a so-called feedback oscillation system or frequency automatically following system. In this embodiment, the control portion 284 may have a part or whole of the function of the ultrasonic oscillator 282. Further, the ultrasonic vibrator 160 allows a high-frequency voltage of 20 to 500 kHz acting on a piezoelectric or magnetostrictive material (such as a piezoelectric element) to be converted into the mechanical vibration of an ultrasonic wave.

Each of the container 260 and the container 280 may be made of a material having nonionic conductivity, electrical insulating property, and/or suitable at least one of plasticity, softness, flexibility, and shape retentivity. Examples of an appropriate material include but are not limited to acryl, polyvinyl chloride, polyacryl, polyamide, polysulfone, polystyrene, polyoxymethylene, polycarbonate, polyester, and copolymers of those materials.

Further, to bring at least an outer surface of the ion exchange membrane 250 of the first polarity of the active electrode assembly 120, at least one surface of the ultrasonic vibrator 160 of the vibrating portion 140, and at least an outer surface of the ion exchange membrane 278 of the second polarity of the counter electrode assembly 130 into contact with an organism, an embodiment may provide the device main body 110 with a handle or other structure that is held and/or pressed by a hand so as to achieve the contact. Alternatively or additionally, the device main body 110 may be adhered to a skin by an adhesive or the like.

As described above for one embodiment of the iontophoresis device 100, at least the ultrasonic vibrator 160 of the vibrating portion 140 is coupled to the active electrode assembly 120 by the vibration absorption materials 180. Accordingly, the vibration is not directly transmitted to the active electrode assembly 120 from the vibrating portion 140, and vibration of the active electrode assembly 120 is suppressed. Therefore, it is possible to prevent (or otherwise reduce) the contact state between the active electrode assembly 120 and the skin from becoming unstable due to vibration. Further in the iontophoresis device 100 of one embodiment, the ultrasonic vibrator 160 is arranged annularly so as to at least partially surround the active electrode assembly 120. Thus, it is possible to more reliably transmit vibration generated by the ultrasonic vibrator 160 to the biological interface coming into contact with the active electrode assembly 120.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An iontophoresis device, comprising:
an active electrode assembly to deliver an active agent to first region of a biological interface in response to applied current;
a counter electrode assembly coupled to the active electrode assembly;
a vibration portion including an oscillator to generate an ultrasonic wave and at least one ultrasonic vibrator coupled to the oscillator and responsive to the ultrasonic wave to generate vibration, having a controllable vibration frequency and a controllable vibration duration, to be applied to a second region of the biological interface different from the first region; and
a vibration absorption material that physically couples the vibration portion to the active electrode assembly, the vibration absorption material adapted to reduce transfer of the vibration from the at least one vibrator to the active electrode assembly so as to stabilize contact between the active electrode assembly and the first region of the biological interface.

2. The device of claim 1 wherein the active electrode assembly is arranged annularly to at least partially surround the at least one vibrator.

3. The device of claim 1 wherein the at least one vibrator is arranged annularly to at least partially surround the active electrode assembly.

4. The device claim 1 wherein the at least one vibrator includes a plurality of vibrators, each of the vibrators having a vibration phase that can be independently controlled.

5. The device of claim 1, further comprising a control unit coupled to the oscillator to control operation thereof and coupled to the active electrode assembly to control application of the current, wherein said control unit is adapted to control said oscillator so as to control said controllable vibration frequency and said controllable vibration duration.

6. The device of claim 1, further comprising a power source coupled to the active electrode assembly and to the vibration portion.

7. The device of claim 1, further comprising:
a first power source coupled to the active electrode assembly; and
a second power source coupled to the vibration portion, the first and second power sources being adapted to facilitate independent control of the active electrode assembly and the vibration portion.

8. The device of claim 1 wherein the vibration portion is detachably coupled to the active electrode assembly.

9. The device of claim 1 wherein said oscillator is controllable so that said vibration also has a controllable vibration intensity.

10. The device of claim 1 wherein said oscillator is controllable so that said vibration also has a controllable timing application, including timing of said vibration to be synchronous with delivery of said active agent to said biological interface.

11. The device of claim 1 wherein said oscillator is controllable so that said vibration also has a controllable timing application, including timing of said vibration to be asynchronous with delivery of said active agent to said biological interface.

12. The device of claim 1 wherein said oscillator is controllable so that said vibration has a vibration characteristic that varies over time.

13. The device of claim 12 wherein said oscillator is controllable so that at least one of said vibration characteristic randomly varies over time.

14. The device of claim 1 wherein said vibration absorption material includes one or more of a rubber pad, a foam pad, and a spring.

15. The device of claim 1 wherein said vibration absorption material is structured as a shock absorber adapted to absorb said vibration, said shock absorber being adapted to absorb said vibration according to at least one of: hysteresis, dry friction, granular spheres, fluid friction, gas compression, magnetism, inertial resistance, composite hydropneumatics, and composite pneumatic springs.

16. A method for an iontophoresis device, the method comprising:
delivering, from an active electrode assembly of the iontophoresis device, an active agent to first region of a biological interface in response to applied current;
delivering vibration having a controllable vibration frequency and a controllable vibration duration to a second region of the biological interface different from the first region without delivering more than a negligible amount of vibration to the first region; and
reducing transfer of the vibration to the active electrode assembly so as to stabilize contact between the active electrode assembly and the first region of the biological interface.

17. The method of claim 16, further comprising:
generating the vibration; and
independently controlling the delivering of the active agent and the generating the vibration.

18. The method of claim 16 wherein delivering the active agent includes delivering the active agent annularly to the first region, the first region having the active agent annularly delivered thereto at least partially surrounding the second region.

19. The method of claim 16 wherein delivering the vibration includes delivering the vibration annularly to the second region, the second region having the vibration annularly delivered thereto at least partially surrounding the first region.

20. The method of claim 16 wherein said controllable vibration frequency and controllable vibration duration are vibration characteristics of said vibration, said vibration characteristics further including controllable vibration intensity, vibration phase, and timing of delivery of said vibration.

21. The method of claim 20 wherein at least one of said vibration characteristics is randomly varied over time.

22. The method of claim 20 wherein said timing of delivery of said vibration is synchronous with delivery of said active agent.

23. An iontophoresis device to administer an active agent to a biological interface, the device comprising:
a power source having a first terminal with a first polarity and a second terminal with a second polarity, the first polarity being opposite to the second polarity;
an active electrode assembly having a first electrode member electrically coupled to the first terminal of the power source, the first polarity being same as a polarity of the active agent, and an active agent reservoir to contain the active agent and being arranged in an electric field generated by the first electrode member;
a counter electrode assembly electrically coupled to the second terminal of the electric power source; and
a vibration portion having an oscillator to provide an ultrasonic wave and at least one vibrator adapted to vibrate in response to the ultrasonic wave provided by the oscillator, wherein vibration generated by the vibrator has a controllable vibration frequency and a controllable vibration duration and is at least partially reduced from being transferred to the active electrode assembly.

24. The device of claim 23 wherein the active electrode assembly further includes:
a first electrolyte solution reservoir to hold an electrolyte solution, the first electrolyte solution reservoir being electrically coupled to the first electrode member;
an ion exchange membrane of the second polarity to selectively pass an ion of the second polarity, the ion exchange membrane and the first electrode member sandwiching the first electrolyte solution reservoir between them; and
an ion exchange membrane of the first polarity to selectively pass an ion of the first polarity, the ion exchange membrane of the first polarity and the ion exchange membrane of the second polarity sandwiching the active agent reservoir between them.

25. The device of claim 24 wherein the counter electrode assembly includes:
a second electrode member electrically coupled to the second terminal of the power source;
a second electrolyte solution reservoir to hold an electrolyte solution, the second electrolyte solution reservoir being electrically coupled to the second electrode member;
an ion exchange membrane of the first polarity to selectively pass an ion having a polarity different from that of the second electrode member, the ion exchange membrane of the first polarity and the second electrode member sandwiching the second electrolyte solution reservoir between them;
a third electrolyte solution reservoir to hold an electrolyte solution, the third electrolyte solution reservoir being placed on a side in the ion exchange membrane of the first polarity that lies opposite to the second electrolyte solution reservoir; and
an ion exchange membrane of the second polarity to selectively pass an ion having same polarity as that of the second electrode member, the ion exchange membrane of the second polarity and the ion exchange membrane of the first polarity sandwiching the third electrolyte solution reservoir between them.

26. The device of claim 23, further comprising a vibration absorption material to couple the vibration portion to the active electrode assembly, the vibration absorption material being adapted to reduce transfer of the vibration from the at least one vibrator to the active electrode assembly so as to stabilize contact between the active electrode assembly and the biological interface.

27. The device of claim 23 wherein the active agent from the active electrode assembly and vibration from the at least one vibrator are applied to different regions of the biological interface.

28. The device of claim 23 wherein the at least one vibrator and the active electrode assembly are in an annular arrangement relative to each other.

29. The device of claim 23, further comprising a control unit coupled to the active electrode assembly and to the vibration portion to independently control the active electrode assembly and the vibration portion, including control of said vibration portion so as to generate said controllable vibration frequency and controllable vibration duration.

30. The device of claim 23 wherein said controllable vibration frequency and controllable vibration duration are vibration characteristics of said vibration, said vibration characteristics further including controllable vibration intensity, vibration phase, and timing of delivery of said vibration.

31. The device of claim 30 wherein at least one of said vibration characteristics is randomly varied over time.

32. The device of claim 30 wherein said timing of delivery of said vibration is synchronous with delivery of said active agent.

33. The device of claim 30 wherein said timing of delivery of said vibration is asynchronous with delivery of said active agent.

34. The device of claim 26 wherein said vibration absorption material includes one or more of a rubber pad, a foam pad, a spring, and a shock absorber.

35. An iontophoresis device, comprising:
an active electrode means for delivering an active agent to first region of a biological interface;
means for generating and delivering vibration having a controllable vibration frequency and a controllable vibration duration to a second region of the biological interface different from the first region; and
means for reducing transfer of the generated vibration to the active electrode means so as to stabilize contact between the active electrode means and the first region of the biological interface.

36. The device of claim 35, further comprising means for independently controlling the active electrode means and the means for generating and delivering vibration, including controlling said controllable vibration frequency and controllable vibration duration.

37. The device of claim 35, further comprising counter electrode means for completing an electrical circuit with the active electrode means.

38. The device of claim 35 wherein the means for reducing transfer of the generated vibration include one or more of at least one rubber pad, at least one foam pad, at least one spring, and at least one shock absorber.

39. The device of claim 35 wherein said controllable vibration frequency and controllable vibration duration are vibration characteristics of said vibration, said vibration characteristics further including controllable vibration intensity, vibration phase, and timing of delivery of said vibration.
